(19) United States
(12) Patent Application Publication
(10) Pub. No.: US 2022/0018840 A1
(43) Pub. Date: Jan. 20, 2022

(54) SYSTEM AND METHOD FOR DETERMINING PRESENCE OF CERTAIN ATTRIBUTES IN A TEST ARTICLE

(71) Applicants: Massoud Akhtari, (US); Niels Akhtari, (US)

(72) Inventors: Massoud Akhtari, Mill Valley, CA (US); Neils Akhtari, Mill Valley, CA (US)

(21) Appl. No.: 17/380,487

(22) Filed: Jul. 20, 2021

Related U.S. Application Data
(60) Provisional application No. 63/054,144, filed on Jul. 20, 2020.

(51) Int. Cl. G01N 33/569; G01N 21/65; G01N 33/543

(52) U.S. Cl. CPC G01N 33/56983; G01N 21/658; G01N 33/54373; G01N 2333/165; G01N 2469/10; G01N 2469/20; G01N 2201/062

(57) ABSTRACT

A method for evaluating a test article provided by a subject for an attribute, such as a medical, industrial, veterinary, agricultural, food, or other attribute, is provided. The design includes providing a cassette comprising at least one ligand selected to match the attribute, applying the test article provided by the patient to the at least one ligand of the cassette, transmitting light energy toward the test article applied to the at least one ligand, sensing optical attributes of light energy provided from the test article applied to the at least one ligand, and providing sensed attributes of the light energy sensed to an electronic device.

The present application claims priority based on U.S. Provisional Patent Application Ser. No. 63/054,144, entitled “System and Method for Determining Presence of Certain Medical Attributes in a Test Article,” inventor Massoud Akhtari, filed Jul. 20, 2020, the entirety of which is incorporated herein by reference.

### BACKGROUND OF THE INVENTION

### Field of the Invention

This present design is directed to systems and methods for determining the presence or absence of particular antibodies, infectious agents such as viruses, and more specifically to systems and methods for determining the presence or absence of SARS/COVID virus and antibodies indicating active infection, past infection or no infection.

### Description of the Related Art

Recent events have illustrated the need for a readily available and accurate testing methodology for the COVID-19 virus. While certain tests are available, those tests are not available for end-to-end individualized breathalyzer home-based use and screening, optional remote sharing, and vary greatly in their accuracies.

The debilitating and fatal effect of SARS-COV-2 virus and the ensuing social and economic shutdown, have shown that improved testing devices are needed to accurately inform individuals and concerned organizations, such as employers, as well as national and international organizations maintaining databases of the infection status of individuals and regions on a daily or as needed basis without the use of swab or other sampling devices and associated discomforts and sampling errors. Individualized assessments would benefit individuals, workplaces, schools, as well as the authorities, of infection status.

Current tests are not for breathalyzer use on on-demand, portable, repeated, and individualized basis and vary in quality, with certain tests reporting less than 50 percent accuracy. Such uncertainties adversely affect the ability to track and treat the disease. COVID-19 is not the only such disease that would benefit from improved testing regimens. Other diseases having different characteristics would benefit from improved testing. Based on multiple reports, these individualized real time (daily, weekly, or as determined) data are needed before educational facilities, workplace economy, and health can return to a functional status as well as in future ongoing basis.

It would therefore be advantageous to offer a testing arrangement or scheme that improves over existing testing regimens, including but not limited to a testing arrangement for COVID-19 and other diseases. It would also be advantageous to provide for disease monitoring in remote areas to promote early detection and prevention of disease outbreaks.

### SUMMARY OF THE INVENTION

The present design provides a method for evaluating a test article provided by an individual for an attribute. In one embodiment the design includes providing a cassette comprising at least one ligand selected with specific affinity for the attribute being tested, applying the test article provided by the individual to the at least one ligand of the cassette, transmitting light energy toward the test article applied to the at least one ligand, sensing optical attributes of light energy provided from the test article applied to the at least one ligand, and providing sensed attributes of the light energy sensed to an electronic device.

In another embodiment, the application of test article to ligand releases light energy from the ligand without the transmission of light energy toward the test article applied to the at least one ligand. This light energy may subsequently be detected by an electronic device.

According to another aspect of the present design, there is provided an apparatus for evaluating a test article provided by a patient for an attribute, comprising a cassette comprising at least one ligand selected to match the attribute, wherein the test article provided by the patient is applied to the at least one ligand of the cassette, a light energy transmitter configured to transmit light energy toward the test article applied to the at least one ligand, a sensor configured to sense optical attributes of light energy provided from the test article applied to the at least one ligand, and an electronic device configured to received sensed attributes of the light energy from the sensor.

According to a further aspect of the present design, there is provided a method for evaluating a test article provided by a patient for an attribute, comprising applying the test article obtained from the patient to at least one ligand selected to match the attribute in a cassette, transmitting light energy toward the test article applied to the at least one ligand, sensing optical attributes of light energy provided from the test article applied to the at least one ligand, and providing sensed attributes of the light energy sensed to an electronic device configured to determine presence or absence of the attribute in the test article.

These and other advantages of the present design will become apparent to those skilled in the art from the following detailed description of the invention and the accompanying drawings.

### BRIEF DESCRIPTION OF THE DRAWINGS

The present design is illustrated by way of example, and not by way of limitation, in the figures of the accompanying drawings and in which like reference numerals refer to similar elements and in which:

FIG. 1 is a general overview of one embodiment of the current design;

FIG. 2 is a representative view of a cassette containing a test article with multiple light sources provided;

FIG. 3 is front view of one embodiment of a cassette and associated hardware operating in accordance with the present design;

FIG. 4 is a side view of the cassette of FIG. 3;

FIG. 5 is a view of the interior of the cassette with a ligand coating to capture the test article (not to scale);

FIG. 6 is a view of the interior of the cassette with a ligand coating and a thin metal film, or positive permittivity material, applied (not to scale);

FIG. 7 is a perspective view of the cassette and associated hardware;

FIG. 8 is a general flowchart of performance of the method according to one embodiment of the present design;

FIG. 9A illustrates a light emitting diode (LED) implementation according to an embodiment of the present design;

FIG. 9B shows the representation of FIG. 9A with altered reflective surface positioning to direct LED light energy toward the bases of individual channels;

FIG. 10 is an antigen affinity detection implementation according to an embodiment of the present design;

FIG. 11 shows power versus peptide concentration;

FIG. 12 shows the divergent power response to current change usable to identify peptides; and

FIG. 13 illustrates the detectability of the binding affinity of a peptide and a target using the device disclosed herein.

### DETAILED DESCRIPTION

The following description and the drawings illustrate specific embodiments sufficiently to enable those skilled in the art to practice the systems and methods described. Other embodiments may incorporate structural, logical, process and other changes. Examples merely typify possible variations. Individual elements and functions are generally optional unless explicitly required, and the sequence of operations may vary. Portions and features of some embodiments may be included in, or substituted for, those of others.

As used herein, the term “subject” refers to any entity receiving or subjected to a test. The term “patient” refers to any entity receiving or registered to receive medical treatment. “Antibody” or “antibodies” refers to both monoclonal and polyclonal antibodies. “Test sample” refers to any item received from a subject, including but not limited to breath items, sputum, and other bodily fluids such as urine, plasma, and so forth. “Test article” refers to the material such as viruses, bacteria, fungi, protozoa, parasites, cellular organisms, cells (e.g., cancer cells, etc.), tissues, antibodies, or other disease-specific molecules or entities being detected within a test sample. “Ligand” refers to molecules or other constructs with affinity for the article being detected on cartridge surface. “Microbials” refer to any small infectious agents such as viruses, bacteria, protozoa, etc. “Medical attribute” or “attribute” is intended broadly to indicate any attribute of the test article, including presence or absence of a virus, health condition, or any quality, feature, trait, or property present in the test article. All of the foregoing terms are intended broadly and use thereof in this document are not intended to be limiting in any way.

The present design comprises an apparatus and method for testing samples and evaluating the samples against known antibodies, antigens, cells, tissues, and/or microbials. The system comprises a cartridge that enables a patient to deposit a sample, also known as a test sample, such as a breath, saliva, sputum, urine, blood, plasma, and other bodily fluid samples, such that the sample can be evaluated against known ligands. The system includes a methodology of illuminating and evaluating the sample provided in the cartridge.

The current design may have a number of applications, including veterinary, industrial, pharmaceutical, agricultural, food industry (e.g. restaurant) applications, as well as in other areas. Ligands are not specifically required in the present design. For example, if the item being examined is a food, drug or other test article, a ligand may be unnecessary. In such a situation, the system disclosed may compare the optical characteristics of a reference or known “good” item with that of the test article to determine if the test article is still viable or includes the attribute being evaluated.

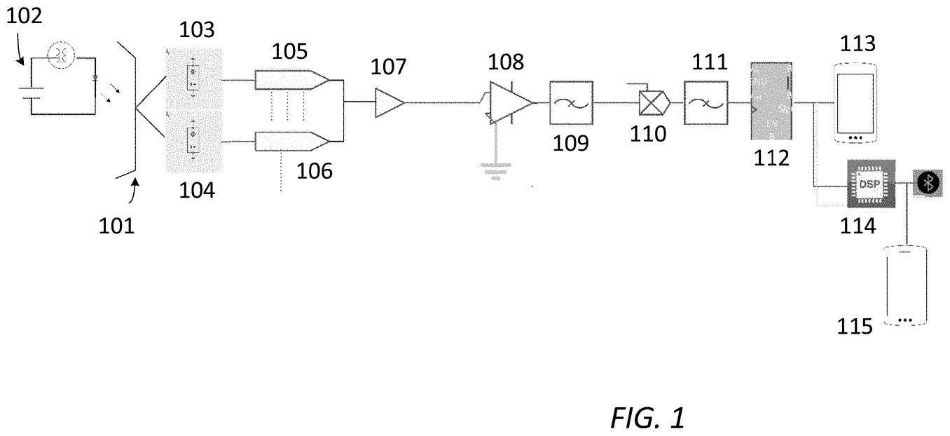

FIG. 1 is a representation of one embodiment of the system. From FIG. 1, cartridge 101 is provided, with a patient sample provided in a desired position within the cartridge. LED or light circuit 102, which may be multiple light sources, directs light energy toward cartridge 101. At least one photodetector with a switching mechanism can be used. In another embodiment, two photodetectors 103 and 104 are provided, one typically being a reference or calibration photodetector, such as photodetector 103. Signals from the photodetectors, such as photodetectors 103 and 104, pass in this representation to analog input elements 105 and 106 and outputs from analog input elements 105 and 106 pass to buffer 107 and gain amplifier 108. A passband filter 109 and anti-aliasing component 111 may be provided to manipulate the signal received, and gain stages 110 may provide gain to the signal. An analog-to-digital (A/D) converter 112 may be provided that converts the analog signals to digital signals, and the raw results may be provided to a computing device, shown as computing device 113 in FIG. 1. Alternately, the output from A/D converter 112 may be provided to DSP 114, which may manipulate the digital data to provide and present the information in an acceptable or desired format and may then present the manipulated data via Bluetooth or other communication method to a computing device 115 or to a remote computing device (not shown).

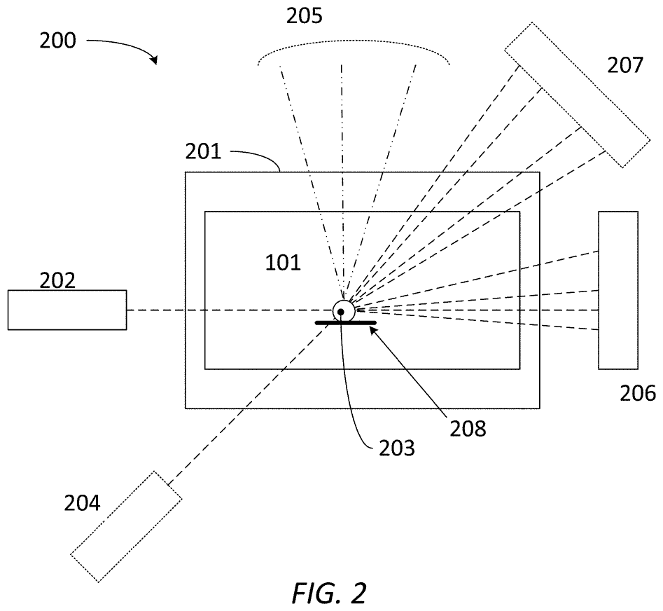

Cartridge 101 may in one embodiment be a removable and disposable ligand cartridge that includes an appropriate ligand with binding affinity for detection of antibodies, cells, tissues, viruses, microbials such as bacteria, peptides, DNA, or RNA, or other disease-specific molecules with light-sensitive attributes or properties. The cartridge may receive a sample from a user, such as by exhaling or blowing into the cartridge. Once the sample is present in the cartridge 101, the cartridge may be provided to examination hardware 200 as shown in FIG. 2. Examination hardware comprises an electronic housing 201 as well as examination components, typically light sources directing light energy toward the sample provided in cartridge 101. In one embodiment, a light emitting device 202 is provided that directs light energy toward the housing and the sample provided. Light emitting device 202 may be a laser or a diode or diodes of desired wavelength or any other light emitting device able to transmit. The light emitting device is of appropriate intensity (picowatt to nanowatt, nanowatt to microwatts, etc.) in both watts and energy based on the test sample and ligand employed, such as a wavelength in any range (including but not limited to 1-99 nm, 100-200 nm, 201-300 nm, 301-400 nm, 401-500 nm, 501-600 nm, 601-700 nm, 701-800 nm, 801-900 nm, 901-1000 nm, 1001-1100 nm, 1101-1200 nm, 1200-3000 nm (IR-A, IR-B, IR-C) i.e. any subrange in the overall range of 1 to 3000 nm,). For example, in one embodiment, the laser diode 202 may transmit at approximately 1 milliwatt and 275 to 285 nm for antibodies, antigens, viruses, bacteria, and so forth. Different specimens being tested for may dictate different intensities, and wavelengths. Further, the depiction in FIG. 2 of light emitting device 202 shows illumination at a particular angle. Other or different angles may be employed, i.e. any subrange between the values of zero to ninety degrees, such as zero to 45, 45 to 90, 30 to 60, and so forth, depending on the type of specimen and the investigation performed.

The system examines test article 203 in cassette 101 in this arrangement. Optional transmitter 204 is shown transmitting at a different angle than light emitting device 202, while optional brightfield light source 205 is also shown. Sensor arrangement 206 receives light energy, such as from light emitting device 202, while optional sensor 207 is also shown that may receive light scattering or transmitted at a different angle.

In one embodiment, a fiber optic waveguide may be employed to transfer desired light wavelength(s) to the cells in cartridge 101. Photodetector(s) may be employed to measure and magnify light once passed or emitted through cells. Illumination in this manner also provides a signal, i.e. light signal, from the doped ligand present in the specimen. If required, light energy can be provided by a different device, such as a bright illumination light source and/or at a different angle, including from above or below the specimen or at a desired angle. The goal is to optically isolate, as best as possible, the target antibodies, viruses, peptides, DNA, RNA, or other molecules or microbials of interest. The system may also employ surface plasmon resonance (SPR) in certain circumstances to improve detection.

Cartridge 101 may be a housing of appropriate material, such as plastic, polymer, and/or glass that houses a zigzag looped conduit configuration. Within the cartridge 101, when a sample is provided, the optical proteomic-doped antigen located proximate to or against the desired test article is adsorbed to the open surfaces of the conduit. Depending on the test being performed and the sample attribute being examined, an individualized cartridge or cartridges may be provided with a particular optical inspection configuration to observe, isolate, and quantify the desired sample attribute or attributes. In operation, a test article is provided in the cartridge 101 or a control cartridge, where the test article may be a control element having known properties when exposed to the light energy. Such a test article may be used for calibration or otherwise establishing a baseline optical reading. One channel of the light energy may therefore be used as a control signal transmission. The cartridge may also be provided with a wash-out port utilized optionally after binding of an antibody, for example.

Cartridge 101 is manufactured such that internal conduit surfaces are coated with ligands with selective affinity to the test article. In the case of a surface plasmon resonance implementation, internal conduits 208 are coated with metal and ligands are adsorbed to this substrate. Once the test article is introduced through breath, saliva, etc., that test article adheres to its ligand counterpart.

The specificity and sensitivity of cartridge 101 can be adapted to employ the most sensitive and specific ligand as they become available. In one embodiment useful in the current environment, spike, surface, and other potential glycoproteins (antigens) associated with SARS-CoV-2 are available. Because monoclonal and polyclonal antibodies to SARS-CoV-2 are produced in response to one or more of these antigens, the corresponding generated antibodies bind to one or more components of this antigen complex. As this antigen library is revised or improved, such revisions are implemented in the cartridge 101. For example, should an improved glycoprotein attribute be determined for a particular virus, the cartridge and associated hardware may be adapted to take advantage of this knowledge.

In one embodiment, the ligand is a monoclonal or polyclonal antibody with specific affinity for a virus, DNA, RNA, cells, tissues, and/or other microbials. In this embodiment, the specific antibody binds to the test article such as SARS-CoV-2 and subsequent optical changes, i.e. changes in the light attributes due to the presence of the test article, can indicate active infection and contagiousness.

In use, the light sources such as a laser or LED may be employed, and detectors measure the light transmitted through the conduits when there is no test article. The system then compares this calibration or reference value to a control transmission. In one embodiment the wavelength (frequency) of the light energy, e.g. LED light, employed is matched to the test article such that the test article has the absorption frequency of the light being used. This matching reduces the level of light that is transmitted in the presence of test article. Such light is measured by photocell detectors 103 and 104, and direct measurement of the light intensity change and differential from control light energy provides two orthogonal measures used to determine the presence of test article(s).

In another embodiment, the system further may optionally assess the presence of test article using a second measure, such as a third orthogonal measure, through introduction of optical proteomic in the peptide sequence of ligand coating. Such an introduction of the optical proteomic results in an additional channel of light resulting from contortion of the ligand upon the ligand coupling with test article. The system measures these various channels, including a channel with no ligand in the cassette 101, certain hardware or control elements provided with the cassette 101, the ligand without optical proteomic, and the ligand with optical proteomic, in the presence and absence of test articles. The differences between these various measurements can provide multiple indicators of the presence or absence of, for example, a particular antibody in the test article or sample provided. Additionally, different light sources, differing in light transmission quality and quantity and/or angle, can provide further information regarding the presence or absence and identification/characterization of the particular test article. A further potential orthogonal measure is introduction of SPR.

Sensitivity of the apparatus increases with addition of each individual orthogonal measurement component to detect pico-molar quantities which is are below the micro-nanomolar concentration of the test article, e.g. in breath, saliva, sputum, urine, plasma, etc.

The various measurements are received by photocell detectors 103 and 104, offering two channels of optical information. Photocell detectors 103 and 104 may be employed multiple times for multiple calibration and/or reference and/or sample evaluations, including application of light energy at different levels and/or angles. The two channels are then buffered and filtered as shown in FIG. 1 and once signal changes are detected, ADC 112 converts output of detectors to the digital domain and may transmit the digital signals to a computing device or processing device. The analog output of the detectors, buffers, and filters, such as those shown in FIG. 1, may be transmitted directly to computing device 113 through a hard-wired connection for analysis. The system may inform a user of infection status (positive or negative) and/or raw digitized results. The information determined may be relayed to a central healthcare facility, such as a clinic or an appropriate system for storage, analysis, and appropriate distribution. Such reporting can be used to manage regional and global disease outbreaks.

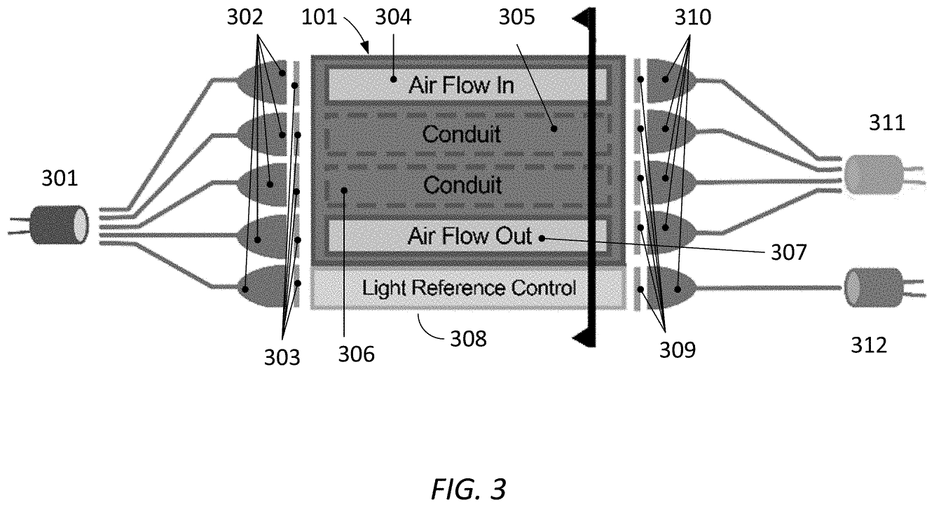

One version of the cassette 101 is shown in FIG. 3. FIG. 3 represents a front view of cassette 101, with light energy coming from the left side and being received at the right side. LED 301 provides light energy via fiber optic waveguides 302. A set of electrochromatic windows 303, one electrochromatic window placed in front of one light transmitter, is also provided. Cassette 101 includes five levels in this embodiment. The first is air flow in level 304, upper conduit level 305, lower conduit level 306, and air flow out level 307. A closed element is shown as light reference control level 308, used as the reference light level. Right side electrochromatic windows 309 are provided with light receiving elements 310 that are connected to, in this view, detector 311 and reference detector 312. These detectors 311 and 312 correspond generally to photocell detectors or photodetectors 103 and 104 in FIG. 1.

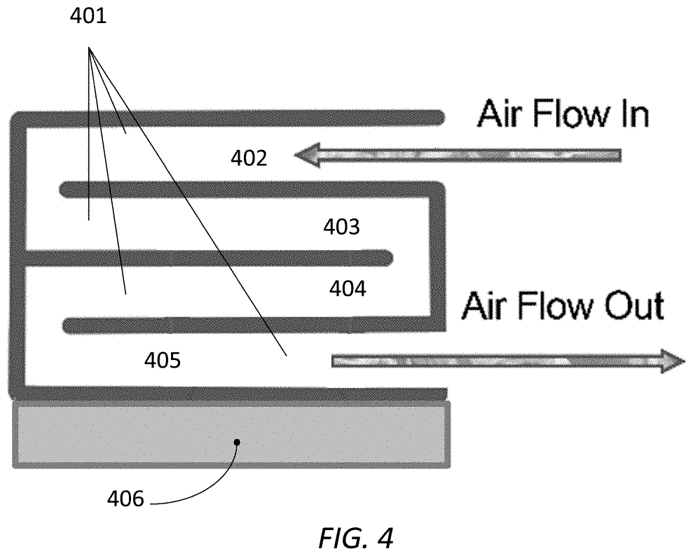

FIG. 4 shows a side view or cutaway side view of cassette 301 shown in FIG. 3. From FIG. 3, a continuous but zigzag channel 401 is provided, with air (fluid) flow in and air (fluid) flow out shown. This arrangement increases the surface area, and each of the four levels shown, levels 402 through 405, correspond to the airflow in level 304, upper conduit level 305, lower conduit level 306, and air flow out level 307. Below these are light reference control channel 406. This type of construction enables samples, in the form of breath, saliva, or other bodily fluid, into the cassette for varying levels of examination with a likelihood that the light energy being transmitted through the cassette can provide satisfactory and beneficial readings of the components therein.

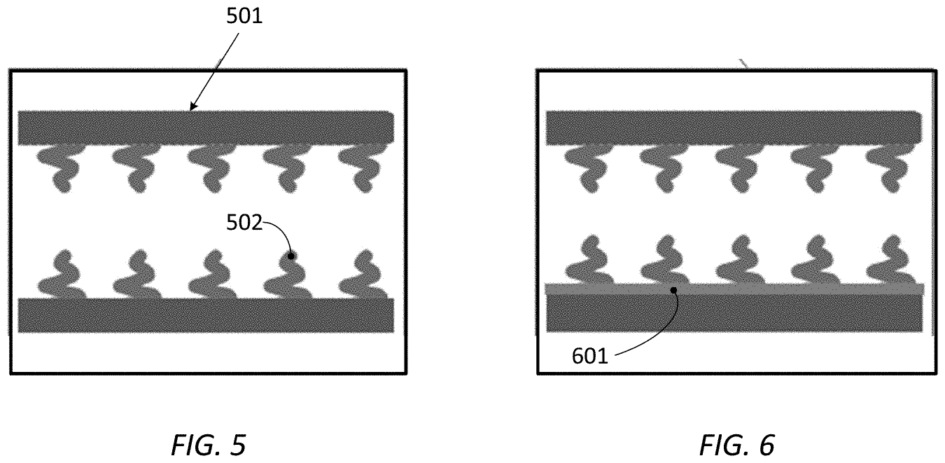

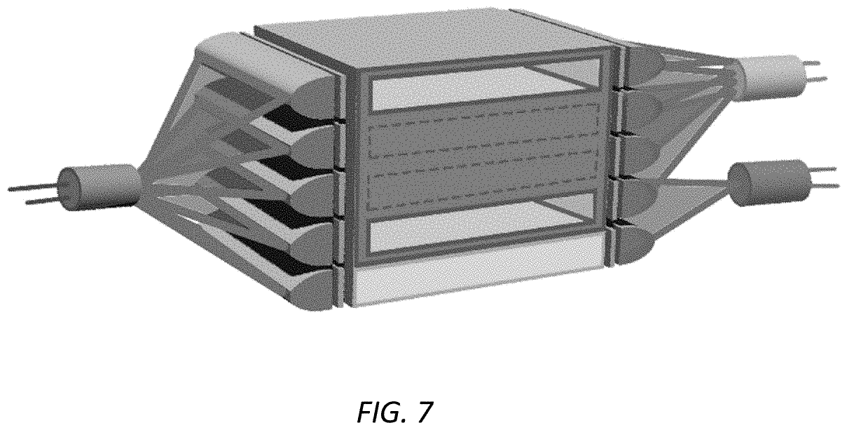

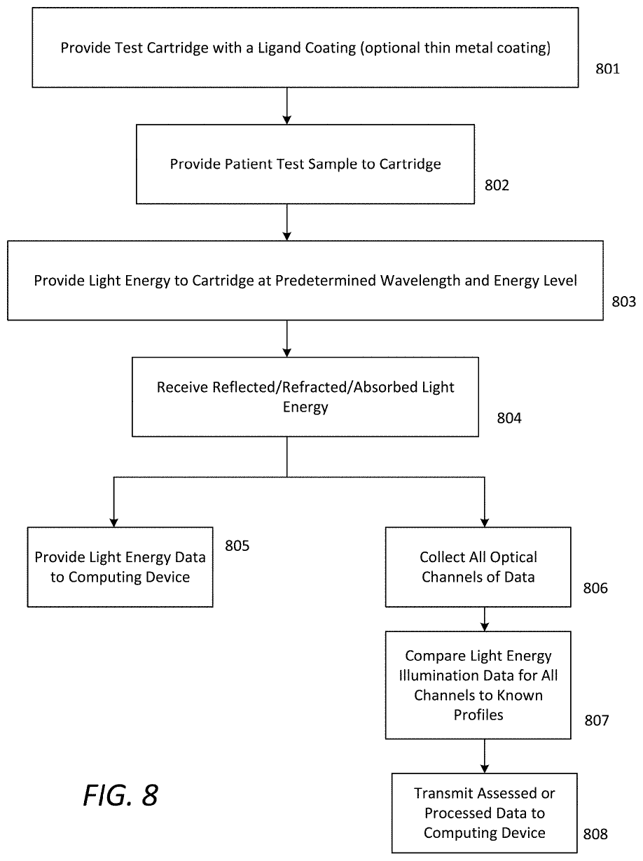

FIGS. 5 and 6 show a detail of the surfaces in the side view of FIG. 4. From FIG. 5, surface 501 has a ligand coating, including ligand 502, in order to capture the sample or test article. In one embodiment, the ligand contains embedded chromatophores or optoproteomics. FIG. 6 illustrates a thin metal film 601 added for surface plasma resonance (SPR). Such elements as shown in FIGS. 5 and 6 may be offered through part or all of a cassette. FIG. 7 illustrates a perspective view of one application of the design.

In operation a patient may blow into, or otherwise his or her spit, mucus, etc. may be provided to, the cassette. Light is transmitted through the light reference control level 308 and is received, providing a control or baseline level of light energy. Light may also be transmitted through one or all of the regions above light reference control level 308. Transmission of light, in the presence of the test article in varying degrees in the cartridge as well as the ligand coating and the optional thin metal film, can cause absorption, scattering, or other optical effects that when received by the detector 311 and processed can indicate presence or absence of an antibody, virus, etc. Other cassette orientations may be employed, including simple surfaces with test articles disposed thereon, in the presence of the ligand coating and/or thin metal film. While shown as a series of linear transmitters in FIG. 7, for example, a series of points may be provided, or light energy directed at an angle, such as downward toward the sample. Absorption, reflection, and refraction may be measured as well as wavelength changes and other optical attributes when the light energy contacts and either passes through or bounces off or otherwise changes when the test sample is exposed to the light energy. Typically, the signals received may be converted to digital values and those digital values may be compared, against one another and/or against known profiles for the test being performed on the test article. In the case of SARS-CoV-2 attributes, a ligand is provided that allows for detection of antibodies to SARS-CoV-2, and when exposed to light energy, the presence or absence of the virus may be detected by the system by comparing the reading or light energy received to known light energy profiles for the virus. As knowledge of the virus improves, the ligand provided may improve. In one embodiment, the ligands are composed of antibodies to SARS-CoV-2 or its nucleic acid components. The ligand selectively captures the virus and the resulting optical changes, i.e. the optical changes between transmission and receipt of the light energy, and optionally the optical changes from the control article, can indicate the presence of virus and a risk of virus transmission.

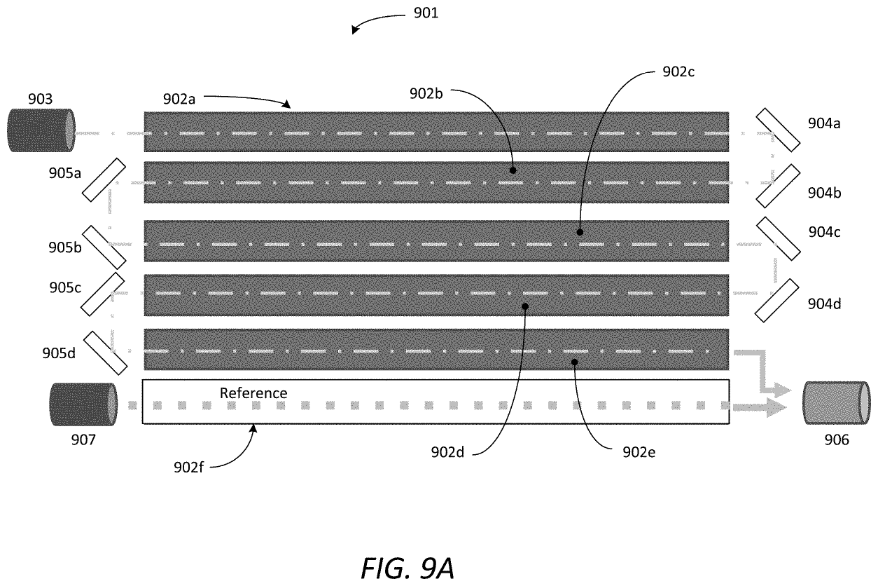

FIG. 8 illustrates a general procedure for operation using the devices provided herein. According to FIG. 8, a test cartridge is provided with at least one of a ligand coating or a thin metal film at point 801. At point 802, a subject test sample is provided to the cartridge. In one embodiment, the cartridge comprises a control region used to calibrate the light energy provided or for use as a reference signal. At point 803, the system provides light energy to the cartridge at a predetermined wavelength and energy level. As noted, the light source may be multiple light sources, and may be laser, LED, brightfield, or other appropriate illumination based on the issue being evaluated. Multiple light sources may be employed, together at the same time or at different times. At point 804, light energy is received, while point 805 indicates the system provides light energy data to a computing device such as a portable smart device for raw data analysis and displaying results such as active infection, prior infection, or no infection. Such receipt of light energy and provision to a computing device may comprise buffering, filtering, and converting of the light energy data, such as performing an analog-to-digital conversion as shown in FIG. 1.

Points 805 as compared with points 806 through 807 represent two options for processing of the data received. Point 806 calls for collecting all channels of optical data. Note that for point 805, multiple channels of data may be provided, collected, and transmitted to the computing device. Point 807 calls for comparing the light illumination data received for all channels to reference and/or to a known database for an assessment of whether the light energy received matches a known profile for the virus or attribute being evaluated. Again, multiple light sources and receptors or sensors may be employed, and different types of light energy may be provided, each potentially yielding different optical attributes. In some instances, exposure to light energy may change the test article, wherein such a change is monitored and evaluated. Each optical attribute may be matched to a reference profile, and the presence or absence of a characteristic of the test article assessed. Point 808 calls for transmitting the result to a computing device, wherein the information provided to the computing device in one embodiment may be presence or absence of the antibodies, the virus, or other microbials of interest or attribute of the test article.

Changed attributes may be rate of absorption of the optical energy provided, density of the ligand, and so forth. Again, improved knowledge regarding a particular virus, for example, may call for changes in examination and determination of presence or absence of properties of the virus. Other attributes may be evaluated as discussed, including but not limited to glycoproteins, antigens, antibodies, RNA, and DNA.

Other devices beyond those showed herein may be used to determine the presence or absence of glycoproteins, antigens, antibodies, RNA, and DNA, and so forth. In one alternate embodiment, the functionality, such as the ligand(s) and other components may be provided within a breathalyzer type apparatus, i.e. a device already set to receive breath of an individual or patient. Such alternate devices may be portable, hand-held, smartphone based (with or without an attachment), and so forth. However, any such apparatus will employ the teachings provided herein.

In any orientation or arrangement, such a system employs a ligand or ligand arrangement that receives a test sample and is exposed to light energy as described herein. The receiving device, or light collector, will receive light energy and post processing determines the presence or absence of the attribute in question. For example, if the system is testing for antibody X, the ligand provided causes antibody X to bond thereto, and when light energy is provided, the received light energy may be altered in a certain way, such as scattered, reflected, absorbed in the test sample such that less light passes through, altered in wavelength, or any other optical alteration of the light detectable to indicate the presence or absence of antibody. The means for collecting the test sample and illuminating the test sample may change, and the net result is the determination, based on light received, of the presence or absence of antibody X based on its receipt by the ligand or ligand arrangement provided. Different antibodies may require different optical exposure and collection protocols.

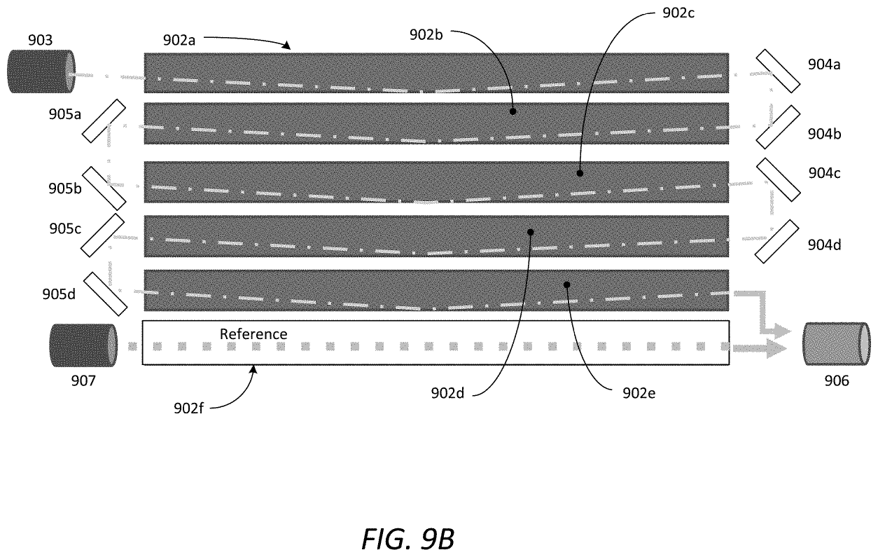

FIG. 9A is a general representation of an alternate embodiment that specifically employs light emitting diodes or LEDs and a series of reflective surfaces. Cassette 901 includes a linear series of channels 902a through 902f, where channel 902f is a reference channel. In this embodiment, the channels enable the transmission of light from LED 903 through the individual channels, being directed off the various mirrors 904a through 904d on the right side and 905a through 905d on the left side. The resultant transmission from bottom channel 902e is provided to both detector 906 and to a waveguide (not shown in this view). Reference transmitter 907 is also an LED transmitter similar or identical to LED 903. Again, as discussed above, a ligand matching the attribute or medical attribute being considered is provided in the channels and the patient may employ the cassette as discussed, with light transmitted as shown and provided to the detector and the waveguide. Presence or absence of the attribute being considered may be determined based on the difference between the reference transmission and that received from the cassette and reflective surface arrangement provided.

While shown as a single LED and a series of reflective surfaces used with six channels, different arrangements may be employed with a different number of channels, more than a single LED transmitter in addition to the reference transmitter, and a different number or arrangement of reflective surfaces. Thus, multiple LED transmitters or multiple reflective surfaces or mirrors may be provided.

While FIG. 9A illustrates path of the light beam transmitted from LED 903 to be largely orthogonal, arrangement of mirrors may allow for different angles to be employed. FIG. 9B illustrates mirrors at slightly different angles such that the light beam passes to a lower portion of each channel. Angles shown in FIG. 9B, both of LED light transmission and reflective surface positioning, are not exact but are representative to illustrate projection toward the bottom of the channels presented. Other orientations may be provided depending on desired region of illumination in a particular channel or channels.

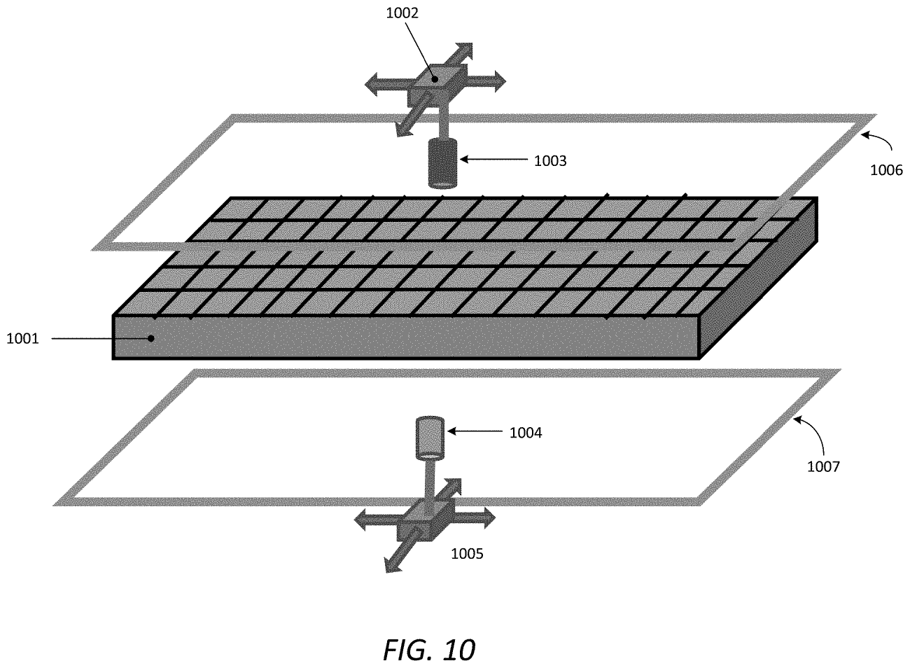

FIG. 10 is a further embodiment according to the present design. The design of FIG. 10 causes light to be transmitted through a clear or see-through tray and received at the other side. In this arrangement, tray 1001 includes clear or see-through lower surfaces. Walls or vertical surfaces separating the various compartments may be provided that may or may not be see-through. “See-through” in the present context means that the light being transmitted toward the see-through element may pass through the see-through element and may be received on the opposite side. In the form presented, a ligand for one or more attributes may be provided in one or more sections of the tray, typically one ligand or component per tray section. Any component other than a ligand that is useful in optically determining the existence or absence of an attribute may be placed in a tray section. Any number of tray sections may be provided. Patient samples may be provided to tray 1001.

Devices presented in FIGS. 3 and 10, for example, may be used in a number of applications, including veterinary, industrial, pharmaceutical, agricultural, food industry (e.g. restaurant) applications, as well as in other areas. In this design, the optical properties of the test article are interrogated via the light source and detector. This interrogation provides information about the viability and quality of the test article, such as whether the food or drug meets a specific standard for use.

In another embodiment, the well tray may include a reflective bottom such that light is reflected up and detected in a detector proximate the light source or integrated in the light source. In this embodiment, only one motor may be required to move the light-source/detector or the tray.

From FIG. 10, the system may either move tray 1001 or may move upper guide motor 1002 having LED 1003 attached thereto as well as providing detector 1004 and lower guide motor 1005. Shown in this view are guide rails 1006 and 1007 for upper and lower guide motors, respectively, contemplating movement of the upper and lower guide motors to position the LED 1003 and detector 1004 over tray 1001 for inspection of the individual tray sections. Alternately, tray 1001 may have a guide motor attached and may move horizontally in the arrangement shown while the LED 1003 and detector 1004 stay in one position. In this arrangement, guide motors for the LED 1003 and detector 1004 are not required or employed. As with other embodiments disclosed herein, light energy is transmitted toward the patient (or other) sample and a component such as a ligand or other indicator while in tray 1001, and the light energy received at detector 1004 may be analyzed to determine the presence or absence of the medical (or other) attribute.

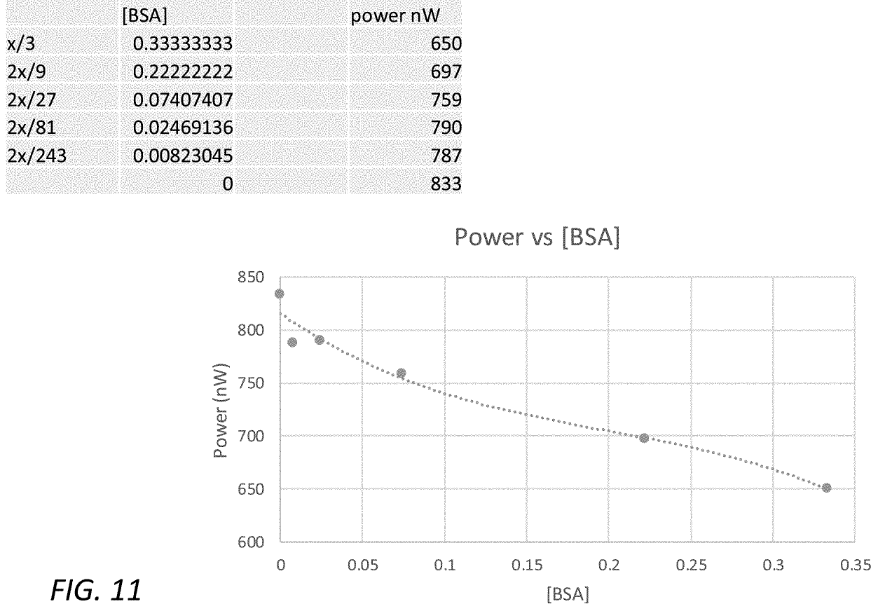

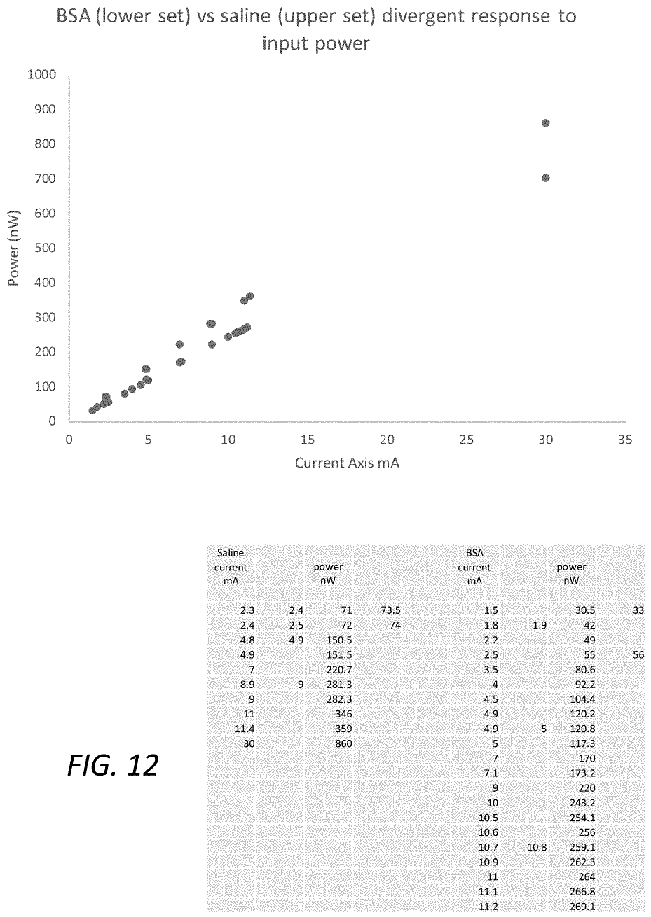

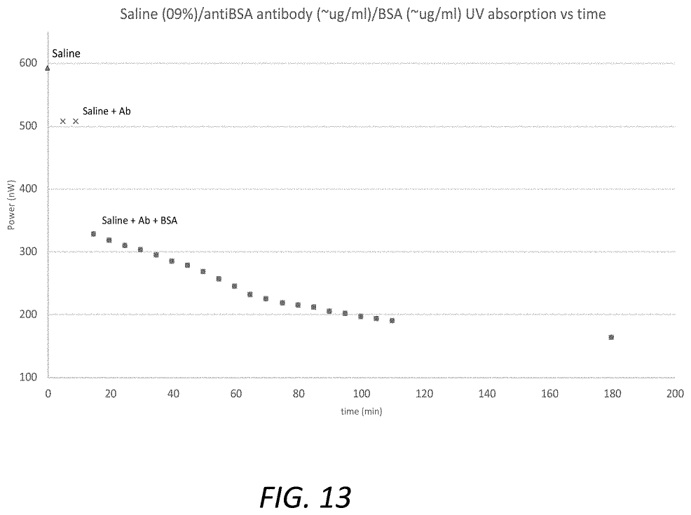

FIGS. 11 to 13 illustrate certain data associated with the present design. FIG. 11 shows power versus peptide concentration, while FIG. 12 shows the divergent power response to current change usable to identify peptides. Applications of the present design may include but are not limited to target identification in drug discovery in oncology, autoimmune disease, vaccine development, as well as an array of other diseases. FIG. 12 represents an experiment performed comparing saline (3 ml) to BSA (bovine serum albumin) solution, with one dust particle of BSA in 3 ml saline. Testing has also been performed on the power response to low current changes, such as 0.5 mA to 11.4 mA, in both saline and BSA solution to check for response characteristics of the system and the BSA solution. FIG. 13 illustrates the detectability of the binding affinity of a peptide and a target using the device disclosed herein.

Thus, according to one aspect of the current design, there is provided a method for evaluating a test article provided by a patient for an attribute is provided. The design includes providing a cassette comprising at least one ligand selected to match the attribute, applying the test article provided by the patient to the at least one ligand of the cassette, transmitting light energy toward the test article applied to the at least one ligand, sensing optical attributes of light energy provided from the test article applied to the at least one ligand, and providing sensed attributes of the light energy sensed to an electronic device.

According to another aspect of the present design, there is provided an apparatus for evaluating a test article provided by a patient for an attribute, comprising a cassette comprising at least one ligand selected to match the attribute, wherein the test article provided by the patient is applied to the at least one ligand of the cassette, a light energy transmitter configured to transmit light energy toward the test article applied to the at least one ligand, a sensor configured to sense optical attributes of light energy provided from the test article applied to the at least one ligand, and an electronic device configured to received sensed attributes of the light energy from the sensor.

The apparatus may allow for evaluating a test article provided by a patient for an attribute or medical attribute or a test article for other applications and indications and may include a cassette comprising at least one ligand selected to match the attribute or medical attribute or any other application or indication.

According to a further aspect of the present design, there is provided a method for evaluating a test article provided by a patient for a attribute, comprising applying the test article obtained from the patient to at least one ligand selected to match the attribute in a cassette, transmitting light energy toward the test article applied to the at least one ligand, sensing optical attributes of light energy provided from the test article applied to the at least one ligand, and providing sensed attributes of the light energy sensed to an electronic device configured to determine presence or absence of the attribute in the test article.

While the present design has been particularly shown and described with reference to some aspects or embodiments, it will be understood by those skilled in the art that various changes in form and detail may be made therein without departing from the spirit and scope of the invention.

It will be appreciated that variations of the above disclosed and other features and functions, or alternatives thereof, can be desirably combined into many other different systems or applications. Also, it will be appreciated that various presently unforeseen or unanticipated alternatives, modifications, variations or improvements therein can be subsequently made by those skilled in the art, which are also intended to be encompassed by the present design. The foregoing description of specific aspects or embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed aspects or embodiments. The phraseology or terminology employed herein is for the purpose of description and not of limitation.

What is claimed is:

1. A method for evaluating a test article provided by a patient for an attribute, comprising:
providing a cassette comprising at least one ligand selected to match the attribute;
applying the test article provided by the patient to the at least one ligand of the cassette;
transmitting light energy toward the test article applied to the at least one ligand;
sensing optical attributes of light energy provided from the test article applied to the at least one ligand; and
providing sensed attributes of the light energy sensed to an electronic device.

2. The method of claim 1, wherein the at least one ligand comprises a ligand coating, and the ligand coating comprises an optical proteomic in a peptide sequence of the ligand coating.

3. The method of claim 1, wherein the cassette comprises a positive permittivity material and the sensing comprises performing an SPR (surface plasmon resonance) evaluation.

4. The method of claim 2, wherein the cassette comprises a positive permittivity material and the sensing comprises performing an SPR (surface plasmon resonance) evaluation.

5. The method of claim 1, further comprising assessing presence or absence of the attribute in the test article after the sensing based on comparison of optical characteristics received the sensing with known optical properties of the attribute.

6. The method of claim 1, wherein multiple light energy sources are employed to illuminate the test article.

7. The method of claim 1, wherein the applying the test article provided by the patient to the at least one ligand of the cassette comprises the patient blowing into the cassette.

8. The method of claim 1, wherein the sensing comprises sensing absorption characteristics of the test article.

9. An apparatus for evaluating a test article provided by a patient for an attribute, comprising:
a cassette comprising at least one ligand selected to match the attribute, wherein the test article provided by the patient is applied to the at least one ligand of the cassette;
a light energy transmitter configured to transmit light energy toward the test article applied to the at least one ligand;
a sensor configured to sense optical attributes of light energy provided from the test article applied to the at least one ligand; and
an electronic device configured to received sensed attributes of the light energy from the sensor.

10. The apparatus of claim 9, wherein the at least one ligand comprises a ligand coating, and the ligand coating comprises an optical proteomic in a peptide sequence of the ligand coating.

11. The apparatus of claim 9, wherein the cassette comprises a positive permittivity material and the sensing comprises performing an SPR (surface plasmon resonance) evaluation.

12. The apparatus of claim 10, wherein the cassette comprises a positive permittivity material and the sensing comprises performing an SPR (surface plasmon resonance) evaluation.

13. The apparatus of claim 9, wherein the electronic device assesses presence or absence of the attribute in the test article based on comparison of optical characteristics received the sensing with known optical properties of the attribute.

14. The apparatus of claim 9, further comprising additional light energy sources configured to illuminate the test article.

15. The apparatus of claim 9, wherein the cassette receives the test article by the patient blowing into the cassette.

16. The apparatus of claim 9, wherein the sensor senses absorption characteristics of the test article.

17. A method for evaluating a test article provided by a patient for an attribute, comprising:
applying the test article obtained from the patient to at least one ligand selected to match the attribute in a cassette;
transmitting light energy toward the test article applied to the at least one ligand;
sensing optical attributes of light energy provided from the test article applied to the at least one ligand; and
providing sensed attributes of the light energy sensed to an electronic device configured to determine presence or absence of the attribute in the test article.

18. The method of claim 17, wherein the at least one ligand comprises a ligand coating, and the ligand coating comprises an optical proteomic in a peptide sequence of the ligand coating.

19. The method of claim 17, wherein the cassette comprises a positive permittivity material and the sensing comprises performing an SPR (surface plasmon resonance) evaluation.

20. The method of claim 17, wherein multiple light energy sources are employed to illuminate the test article.

21. The method of claim 17, wherein the sensing comprises sensing absorption characteristics of the test article.

22. The method of claim 17, wherein said light energy is provided by a light emitting diode (LED).

23. The apparatus of claim 9, further comprising an arrangement of reflective surfaces employed with the cassette.
